(19) United States
(12) Patent Application Publication
(10) Pub. No.: US 2015/0367005 A1
(43) Pub. Date: Dec. 24, 2015

(54) METHOD OF SYNTHESIZING FLUORINE-18 LABELED RADIOPHARMACEUTICALS IN ETHANOL AND WATER

(71) Applicant: THE REGENTS OF THE UNIVERSITY OF MICHIGAN, Ann Arbor, MI (US)

(72) Inventors: Peter J. H. Scott, Ypsilanti, MI (US); Megan N. Stewart, Ann Arbor, MI (US); Brian Hockley, Superior Township, MI (US)

(73) Assignee: THE REGENTS OF THE UNIVERSITY OF MICHIGAN, Ann Arbor, MI (US)

(21) Appl. No.: 14/744,627

(22) Filed: Jun. 19, 2015

Related U.S. Application Data
(60) Provisional application No. 62/014,304, filed on Jun. 19, 2014.

(51) Int. Cl. A61K 51/04; C07H 5/02; C07C 309/73; C07B 59/00

(52) U.S. Cl. CPC A61K 51/0491; C07B 59/005; C07B 59/001; C07H 5/02; C07C 309/73; A61K 51/0497; A61K 51/04; C07B 2200/05

(57) ABSTRACT

A method of preparing fluorine-18 labeled radiopharmaceuticals (¹⁸F-radiopharmaceuticals) is disclosed. More particularly, the present invention relates to a method of preparing an ¹⁸F-radiopharmaceutical by reacting a salt containing fluorine-18 (¹⁸F) with an alkyl halide or an alkyl sulfonate in the presence of water and ethanol to obtain a high yield of the ¹⁸F-radiopharmaceutical. The synthetic method eliminates the use of toxic and/or environmentally-unfriendly organic solvents.

### CROSS-REFERENCE TO RELATED APPLICATIONS

This application claims the benefit of U.S. provisional patent application No. 62/014,304, filed Jun. 19, 2014, incorporated herein by reference in its entirety.

### FIELD OF THE INVENTION

The present method relates to fluorine-18-labeled radiopharmaceuticals (¹⁸F-radiopharmaceuticals) and their method of preparation. More particularly, the present invention relates to the preparation of an ¹⁸F-radiopharmaceutical by reacting a fluorine-18 (¹⁸F) salt with an alkyl halide or alkyl sulfonate in a water/ethanol solvent, and in the absence of toxic and/or environmentally unfriendly organic solvents, to provide the ¹⁸F-radiopharmaceutical in high radiochemical yields (RCY).

### BACKGROUND OF THE INVENTION

Generally organofluoro compounds are prepared via a substitution reaction by reacting an alkyl halide or alkyl sulfonate with a fluorine salt as shown below.

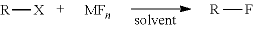

The halide (X) of the alkyl halide is Cl, Br, or I. The sulfonate (X) of the alkyl sulfonate is SO₃R² wherein R² is alkyl or aryl group. M is a cation for the fluoride ion (F). The alkyl group preferably is a C₁-C₁₂ alkyl halide or C₁-C₁₂ alkyl sulfonate. For example, the alkyl sulfonate is selected from the group consisting of methyl sulfonate, ethanyl sulfonate, isopropyl sulfonate, chloromethyl sulfonate, trifluoromethyl sulfonate, and chloroethyl sulfonate. The aryl group preferably is selected from the group consisting of phenyl, C₁-C₄ alkylphenyl, halophenyl, C₁-C₄ alkoxyphenyl, and nitrophenyl. Nonlimiting examples are methylphenyl sulfonate, ethylphenyl sulfonate, chlorophenyl sulfonate, bromophenyl sulfonate, methoxyphenyl sulfonate, and nitrophenyl sulfonate.

The fluoride salt (MFₙ), as a source of fluoride ion, is selected from the group consisting of an alkali metal fluoride containing alkali metals such as lithium, sodium, potassium, rubidium, or cesium; an alkaline earth metal fluoride containing alkaline earth metals such as magnesium, calcium, strontium, or barium; and ammonium fluorides containing ammonium or a derivative, such as a tetraalkylammonium.

Nucleophilic fluorination reaction is carried out in a polar aprotic solvent, such as acetonitrile (CH₃CN), DMF, DMSO, to increase the solubility of fluoride salt and the reactivity of the fluoride ion. It is known in the art that an alcohol (a protic solvent) is not suitable in the nucleophilic fluorination reaction. It is further known that an alcohol forms hydrogen bonds with fluoride ion, and thereby reduces fluoride ion reactivity in the nucleophilic fluorination reaction.

The preparation of an ¹⁸F-radiopharmaceutical therefore includes two steps. The first step is to provide a mixture of an ¹⁸F-containing salt, typically ¹⁸F-labeled potassium fluoride ([18F]KF) and a crown ether, such as Kryptofix-2.2.2 (K222). The [18F]KF/K222 mixture is dried from a water-acetonitrile azeotrope. In the second step, labeling of the alkyl halide or alkyl sulfonate with the dried [18F]KF/K222 mixture is conducted in an organic solvent, such as N, N-dimethylformamide (DMF), dimethysulfoxide (DMSO), or tetrahydrofuran (THF), for example.

The use of such organic solvents in the drying step and the labeling step is expensive, and the organic solvents that pose toxicity and environmental concerns, in addition to disposal problems.

FDG, which is also known as fludeoxyglucose, fluorodeoxyglucose, [¹⁸F]FDG, and 2-deoxy-2-(¹⁸F)fluoro-D-glucose, is a radiopharmaceutical which is widely used in nuclear medicine for diagnostic studies, such as those using Positron Emission Tomography (PET) body scanning. FDG contains the positron-emitting radioactive isotope of fluorine-18. After the injection of FDG in a patient, a PET scanner forms images of the FDG in the body, and these images can be used to diagnose number of medical indications. FDG has the following chemical structure:

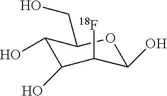 [SMILES: OCC1OC(O)C([18F])C(O)C1O]

The present invention is directed to overcoming the problems associated with the organic solvents used in the preparation of ¹⁸F-radiopharmaceuticals.

### SUMMARY OF THE INVENTION

The present invention is directed to a method of preparing ¹⁸F-radiopharmaceuticals. In particular, the present method overcomes problems encountered in prior methods of preparation that utilized organic solvents in the drying of an ¹⁸F salt/crown ether mixture and in the labeling of an alkyl halide or alkyl sulfonate using the mixture.

The present invention is directed to a method of preparing an ¹⁸F-radiopharmaceutical comprising:
(a) providing a mixture of an ¹⁸F-containing salt and a crown ether;
(b) drying the mixture of (a) using a water-ethanol azeotrope; and
(c) reacting the dried product of (b) with an alkyl halide or alkyl sulfonate, wherein a solvent used in the reaction consists essentially of water and ethanol,
to provide the ¹⁸F-radiopharmaceutical.

### DETAILED DESCRIPTION OF THE PREFERRED EMBODIMENTS

A typical method of preparing an ¹⁸F-radiopharmaceutical utilizes [¹⁸F]KF, in which the fluoride source is generated through an anion exchange between, for example, potassium carbonate (K₂CO₃) and a cyclotron-produced [¹⁸F]-containing species, which often requires the addition of an aza-crown ether, e.g., Kryptofix®2.2.2 (4,7,13,16,21,24-hexaoxa-1,10-diazabicyclo[8.8.8]-hexacosane) to enhance reactivity. While suitable for production of clinical quantities, the moderate efficiency, demanding purification, and complex implementation of such method may not be suitable for widespread commercial application.

In addition, the resulting [¹⁸F]KF/K222 mixture is dried using a water/acetonitrile azeotrope. The resulting dried mixture is reacted with an alkyl halide or alkyl sulfonate, using an organic solvent, to yield the ¹⁸F-radiopharmaceutical. The drying step and subsequent labeling reaction utilizes organic solvents that are expensive, toxic and/or environmentally unfriendly, and that require disposal.

The present invention provides a method that eliminates the use and disposal of hazardous organic solvents. The elimination of hazardous organic solvents in the pharmaceutical industry provides rewards both economically and in environmental safety.

It has been found that organic solvents can be eliminated in the preparation of ¹⁸F-radiopharmaceuticals used in Positron Emission Tomography (PET) imaging. The use of PET imaging to non-invasively image biochemical processes in living human subjects is being increasingly applied to personalized medicine in the academic setting, and to drug discovery in the pharmaceutical industry. In this procedure, patients receive an injection of a radiopharmaceutical (i.e., a bioactive molecule typically radiolabeled with a short half-life radionuclide, such as carbon-11 or fluorine-18) followed by PET imaging of the radioactivity distribution in the body. In particular, fludeoxyglucose (FDG) synthesized from ¹⁸F is commonly used in Positron Emission Tomography (PET) medical imaging. Reflecting the increased global demand for access to PET imaging, sophisticated reactions for the synthesis of radiopharmaceuticals continue to be developed.

Radiopharmaceuticals labeled with ¹⁸F are among the most prevalent because the long half-life of ¹⁸F (i.e., 109.77 min) allows sophisticated radiochemistry and distribution of the resulting ¹⁸F-radiopharmaceuticals to satellite PET centers lacking a cyclotron. However, the art and science of ¹⁸F-radiopharmaceutical synthesis present unique challenges. First and foremost, the half-life still demands fast and efficient radiochemical syntheses, frequently in a radiochemistry laboratory adjacent to the hospital PET scanner. Secondly, radiopharmaceutical doses must be formulated and the quality control procedures finished rapidly because administration to a patient typically occurs within one hour following the end of synthesis (EOS). Therefore, a streamlined quality control (QC) is an essential aspect of ¹⁸F-radiopharmaceutical production. Despite these challenges, a large number of ¹⁸F-radiopharmaceuticals have been developed for imaging applications in neurology, oncology, and cardiology.

Each ¹⁸F-radiopharmaceutical used in imaging applications for human studies is synthesized by ¹⁸F-fluorination of the corresponding precursor using [18F]KF in combination with Kryptofix-2.2.2 (K222) as a phase transfer catalyst. The [18F]fluoride/K222 mixture is dried from a water-acetonitrile azeotrope, and the subsequent labeling reaction conducted in an organic solvent, such as N,N-dimethylformamide (DMF), dimethylsulfoxide (DMSO), acetonitrile (MeCN), or tetrahydrofuran (THF). Recently however, additional scrutiny over the supply and disposal of such solvents has made their routine use increasingly problematic. Furthermore, as the field of radiochemistry becomes increasingly regulated, the use of such solvents make quality control (QC) of radiopharmaceuticals more challenging and time consuming.

For QC purposes, the solvent classification system outlined by the International Conference on Harmonisation of Technical Requirements for Registration of Pharmaceuticals for Human Use (ICH) is used. Solvents are classified as Class I, II, or III, depending upon their toxicity. Class I solvents (e.g., benzene, carbon tetrachloride) are the most toxic, have single digit ppm dose limits, and typically are not ever employed in radiopharmaceutical syntheses. Class II (e.g., MeCN, DCM, DMF, THF) and Class III solvents (e.g., ethanol, DMSO, acetone, methyl ethyl ketone) are widely used in radiopharmaceutical syntheses. Class II solvents are considered to have intermediate toxicity, reflected in residual solvent limits of, typically, a few hundred ppm per dose. Class III solvents are generally considered safe reflected in the 5000 ppm dose limit suggested by ICH. Presently, residual solvent analysis of all Class II and III solvents employed in a radiopharmaceutical synthesis (i.e., for module cleaning, synthesis, and purification) are performed. However, a typical radiopharmaceutical production program has seen continuous and rapid growth in recent years such that performing two or three fluorine-18 syntheses, up to four carbon-11 syntheses, and up to two nitrogen-13 syntheses every day for clinical purposes is usual. Because each synthesis requires eight gas chromatography (GC) runs (blank injection, 3 reference standard injections, blank injection, 3 dose injections), and each run takes 20-30 minutes when post-run instrument cooling is factored in, scheduling conflicts and a significant bottleneck in the clinical production and QC operations is created.

Because of the short half-lives of imaging radiopharmaceuticals suitable for human use (on the order of hours or less), multiple syntheses are required each day at or near PET imaging facilities. Each synthesis produces waste solvent and requires strict quality controls for safe solvent levels that are increasingly expensive to handle.

In an effort to eliminate class II solvents from ¹⁸F-radiopharmaceutical products, various methods of synthesis were investigated, including the use of water and ethanol, and in the absence of other solvents, throughout the synthetic process. Ethanol was tested because ethanol often is a component of purification and formulation systems, can be purposefully added to inhibit oxidative radiolysis, and from a toxicity standpoint is an acceptable solvent choice. Ethanol is also a sustainable solvent suitable for green chemistry.

The use of an aqueous ethanol solvent throughout the ¹⁸F-radiopharmaceutical preparation required development of a method for drying an [18F]fluoride from an aqueous ethanol azeotrope, and conducting the subsequent labeling reaction in ethanol and water. Historically however, radiosyntheses in alcohol solvents and/or water have always been considered incompatible with [18F]fluorination reactions because alcohols and water were thought to retard SN₂ reactions due to an unfavorable solvation of the fluoride.

It was found that replacing acetonitrile with ethanol in the drying of [¹⁸F]KF/K222 mixture did not lead to any adverse effects. Surprisingly, using an aqueous ethanol solvent in the labeling reaction of an alkyl halide or alkyl sulfonate readily provided ¹⁸F-radiopharmaceuticals in high radiochemical yields (RCY). The present method is not limited to a particular ¹⁸F-containing salt or an ¹⁸F-radiopharmaceutical.

Therefore, in various embodiments, the ¹⁸F source includes, but is not limited to, ¹⁸F-labeled alkali metal fluorides and alkaline earth metal fluorides (e.g., ¹⁸F lithium fluoride, ¹⁸F sodium fluoride, ¹⁸F potassium fluoride, ¹⁸F rubidium fluoride, ¹⁸F cesium fluoride, ¹⁸F beryllium fluoride, ¹⁸F magnesium fluoride, ¹⁸F calcium fluoride, ¹⁸F strontium fluoride, ¹⁸F barium fluoride), ¹⁸F-labeled ammonium fluorides (e.g., ¹⁸F-labeled tetraalkylammonium fluorides, such as ¹⁸F tetramethylammonium fluoride, ¹⁸F tetraethylammonium fluoride, ¹⁸F tetrapropylammonium fluoride, ¹⁸F tetrabutylammonium fluoride, tributylammonium fluoride; secondary ammonium fluorides, including dibutylammonium fluoride and dihexylammonium fluoride; and primary ammonium fluorides, including butylammonium fluoride and hexylammonium fluoride), and complexes thereof with complexing compounds such as crown ethers (e.g., complexes with 12-crown-4, 15-crown-5, 18-crown-6, dibenzo-18-crown-6, and diaza-18-crown-6), for example, ¹⁸F potassium fluoride.18-crown-6 complex.

The ¹⁸F-radiopharmaceutical can be ¹⁸F-labelled fludeoxyglucose, flubatine, fluoropropylcarbomethoxytropane (FP-CT), fluorothymidine, fluorocholine, fluoroethylcholine, fluoropropylcholine, fluoro-2-dialkylamino-6-acylmalonodinitrile naphthalene (FDDNP), fluoroisonidazole, fluoroestradiol, and fluoroethyl tosylate, for example.

The solvent used in the drying step for the ¹⁸F-containing salt/crown ether mixture and in the labeling reaction of the alkyl halide or alkyl sulfonate consists essentially of water and ethanol. As used herein, the term “consisting essentially of” means that the solvent contains at least 99%, at least 99.5%, and preferably at least 99.9% of water and ethanol, and any remaining solvent is a Class III solvent as defined by the ICH. The Class III solvent is not intentionally added to the solvent, but is present as an inadvertent impurity or additive. In a preferred embodiment, the solvent is 100% water and ethanol, i.e., the solvent consists of water and ethanol.

The amount of ethanol in the aqueous ethanol solvent can be any amount from 0-100% by volume, but is typically 75% to 95%, by volume; preferably 80% to 90%, by volume; and more preferably 82% to 90%, by volume.

To demonstrate the present method, the replacement of acetonitrile with ethanol in the drying step of the [¹⁸F]KF/K222 mixture had no detrimental effect on the process. [18F]fludeoxyglucose (FDG) and [18F]fluoroethyltosylate were prepared using this modified drying step and the traditional solvents used in a fluorination reaction.

Then, fluorination reactions were performed using ethanol and/or water. The reactions were found to proceed smoothly. [18F]Fludeoxyglucose (FDG) was employed as a model substrate, and [18F]KF, potassium carbonate and Kryptofix 2.2.2 were used as standard fluorination conditions. Replacing acetonitrile with ethanol in the azeotropic fluoride-drying step, followed by the synthesis of FDG using ethanol and water as the reaction solvent was performed. The effects of reaction solvent, time, temperature, and stoichiometry on the radiochemical yield of FDG were investigated using a matrix approach.

Replacement of acetonitrile by ethanol in the azeotropic drying of fluoride had no detrimental effect on the process. Mannose triflate then was dissolved in aqueous ethanol, and reacted with ¹⁸F to generate protected FDG in 63% radiochemical yield (RCY). Adding water to the reaction solvent improved precursor solubility and led to increased product yields. A reaction solvent containing 15% water:85% ethanol led to 63% RCY of protected-FDG. Near quantitative hydrolysis using 1M NaOH, followed by standard Sep-Pak purification, provided doses of [18F]FDG that confirmed established specifications.

For example, in various tests, the radiochemical yields (RCY) of protected [18F]FDG were as follows (using 4-40 mg precursor and 0.7-2.0 mL of reaction solvent):

3% water in ethanol—3% RCY;

5% water in ethanol—6% RCY; and

15% water in ethanol—42% RCY.

In addition to [¹⁸F]FDG, [18F]fluoroethyl tosylate and [18F]flubatine also were synthesized using only ethanol and water throughout the entire manufacturing process.

The following examples illustrate the preparation of ¹⁸F-radiopharmaceuticals using ethanol and water as solvents, and in the absence of the organic solvents.

### EXAMPLES

### Example 1

### Synthesis of [¹⁸F]KF.18-Kryptofix-2.2.2 Complex

Potassium [¹⁸F]fluoride was prepared using a TRACERLab FXFN automated radiochemistry synthesis module (General Electric, GE). All loading operations were conducted under ambient atmosphere. Argon was used as a pressurizing gas during automated sample transfers. [¹⁸F]Fluoride was produced via the ¹⁸O(p,n)¹⁸F nuclear reaction using a GE PETTrace cyclotron (40 μA beam for 2 min generated ca. 150 mCi of [¹⁸F]fluoride). The [¹⁸F]fluoride was delivered to the synthesis module in a 1.5 mL bolus of [¹⁸O]water and trapped on a QMA-light Sep-Pak to remove [¹⁸O]water. [¹⁸F]Fluoride was eluted into the reaction vessel using aqueous potassium carbonate (1-3.5 mg in 0.5 mL of water). A solution of Kryptofix-2.2.2 (10-15 mg in 1 mL of ethanol) was added to the reaction vessel, and the resulting solution was dried by azeotropic distillation to give dry [¹⁸F]KF.Kryptofix-2.2.2. Evaporation was achieved by heating the reaction vessel to 100° C. and drawing vacuum for 4 min. After this time, the reaction vessel was subjected to an argon stream and simultaneous vacuum draw for an additional 4 min. The resulting fluoride either was used in the automated synthesis module or was transferred to a sterile vial for subsequent use in manual reactions.

### Example 2

### General Procedure for Manual Synthesis of ¹⁸F-Labeled Compounds using Dried [18F]Fluoride

On the bench top, dried [¹⁸F]Fluoride (from [0040]) was re-solubilized in the reaction solvent (ethanol or a mixture of ethanol and water), then added to a solution of the precursor, also dissolved in the reaction solvent (ethanol or a mixture of ethanol and water). In some cases, heating was required to dissolve the precursor in ethanol/water solution. The sealed reaction vessel was placed in a sand bath at 100° C. with stirring for 30 minutes. Post reaction, the vessel was allowed to cool for 5-10 minutes and vented prior to analysis via radio-TLC.

### Example 3

### General Procedure for Automated Synthesis of ¹⁸F-Labeled Compounds using Dried [¹⁸F]Fluoride

The production-scale synthesis of fluorine-18 labeled radiotracers was conducted using a TRACERLab FXFN automated radiochemistry synthesis module (General Electric, GE). The synthesis module was pre-charged with a solution of the appropriate precursor in the reaction solvent (ethanol or a mixture of ethanol and water) to be added from an automated port prior to ¹⁸F delivery. [¹⁸F]Fluoride was produced via the ¹⁸O(p,n)¹⁸F nuclear reaction using a GE PETTrace cyclotron (40 μA beam for 30 min generated 1,500 mCi of [¹⁸F]fluoride). The [¹⁸F]fluoride was delivered to the synthesis module (in a 1.5 mL bolus of [¹⁸O]water) and trapped on a QMA-light Sep-Pak to remove [¹⁸O]water. [¹⁸F]Fluoride was eluted into the reaction vessel using a solution of aqueous potassium carbonate (1 mg in 0.5 mL of water) and Kryptofix-2.2.2 (10 mg in 1 mL of ethanol). The resulting solution was dried by azeotropic distillation to give dry [¹⁸F]KF.Kryptofix-2.2.2. Evaporation was achieved by heating the reaction vessel to 100° C. and drawing vacuum for 5 min. After this time, the reaction vessel was subjected to an argon stream and simultaneous vacuum draw for an additional 5 min. The reaction vessel was cooled to 50° C., and the preloaded ethanol-containing solution of the appropriate precursor was added to the reaction vessel; the vessel was then sealed, heated to 100° C., and held at that temperature for 30 min. The reaction vessel was then cooled to 50° C. and transferred to a sterile vial for analysis (radio-TLC and radio-HPLC).

### Example 4

### General Procedure for Manual Synthesis of ¹⁸F-Labeled Compounds without Drying the [¹⁸F]Fluoride

[¹⁸F]Fluoride was produced via the ¹⁸O(p,n)¹⁸F nuclear reaction using a GE PETTrace cyclotron (40 μA beam for 1 min generates 80-100 mCi of [¹⁸F]fluoride). The [¹⁸F]fluoride was delivered to the synthesis module (in a 1.5 mL bolus of [¹⁸O]water) and trapped on a QMA-light Sep-Pak to remove [¹⁸O]water. [¹⁸F]Fluoride was eluted into the reaction vessel using an ethanolic solution of potassium carbonate (1 mg) and Kryptofix-2.2.2 (10 mg) in the appropriate reaction solvent (ethanol or a mixture of ethanol and water). This was eluted into the reaction vessel containing a solution of the precursor, also dissolved in the reaction solvent (ethanol or a mixture of ethanol and water). In some cases heating was required to dissolve the precursor in ethanol/water solution. The sealed reaction vessel was placed in a sand bath at 100° C. with stirring for 30 minutes. Post reaction, the vessel was allowed to cool for 5-10 minutes and vented prior to analysis via radio-TLC.

### Example 5

### Manual Synthesis of [¹⁸F]Fludeoxyglucose using Dried Fluoride

[¹⁸F]Fludeoxyglucose was prepared by the general procedure for manual synthesis of ¹⁸F-labeled compounds described in Example 2, a range of reaction conditions were demonstrated to be effective, as summarized in Table 1 below.

TABLE 1

| Solvent | Precursor Amt | Reaction Conditions | Yield |
|---|---|---|---|
| 3% H₂O/EtOH | 8 mg | 100° C., 30 min | 3% |
| 5% H₂O/EtOH | 4 mg | 100° C., 30 min | 6% |
| 10% H₂O/EtOH | 8 mg | 100° C., 30 min | 7% |
| 15% H₂O/EtOH | 40 mg | 100° C., 30 min | 40% (n = 4) |

### Example 6

### Automated Synthesis of [¹⁸F]Fludeoxyglucose using Dried Fluoride

The synthesis of [¹⁸F]fludeoxyglucose was conducted using a TRACERLab FXFN automated radiochemistry synthesis module (General Electric, GE). The synthesis module was pre-charged with a solution of the manose trifflate in the reaction solvent (85% ethanol:15% water) to be added from an automated port prior to ¹⁸F delivery. [¹⁸F]Fluoride was produced via the ¹⁸O(p,n)¹⁸F nuclear reaction using a GE PETTrace cyclotron (40 μA beam for 30 min generates 1,500 mCi of [¹⁸F]fluoride). The [¹⁸F]fluoride was delivered to the synthesis module (in a 1.5 mL bolus of [¹⁸O]water) and trapped on a QMA-light Sep-Pak to remove [¹⁸O]water. [¹⁸F]Fluoride was eluted into the reaction vessel using aqueous potassium carbonate (1 mg in 0.5 mL of water). A solution of Kryptofix-2.2.2 (10 mg in 1 mL of ethanol) was added to the reaction vessel, and the resulting solution was dried by azeotropic distillation to give dry [¹⁸F]KF.Kryptofix-2.2.2. Evaporation was achieved by heating the reaction vessel to 100° C. and drawing vacuum for 5 min. After this time, the reaction vessel was subjected to an argon stream and simultaneous vacuum draw for an additional 5 min. The reaction vessel was cooled to 50° C., and the preloaded ethanol-containing solution of the appropriate precursor was added to the reaction vessel, and the vessel was sealed, heated to 100° C., and held at that temperature for 30 min. The reaction vessel was then cooled to 50° C., and 2N NaOH (1 mL) was added. The resulting solution was diluted and neutralized with a HCl/Citrate solution (5 mL) and transferred to a sterile vial for analysis (radio-TLC and radio-HPLC). Under automated conditions, mannose triflate was converted to [¹⁸F]fludeoxyglucose in a radiochemical yield (RCY) of 35% (non-decay corrected yield at end-of-synthesis based upon [¹⁸F]fluoride).

### Example 7

### Automated Synthesis of [¹⁸F]Fludeoxyglucose using Non-Dried Fluoride

[¹⁸F]Fludeoxyglucose was prepared by the general procedure for automated synthesis of ¹⁸F-labeled compounds (Example 6). In this case the [¹⁸F]Fluoride was eluted from a QMA cartridge into the reaction vessel using 15% water:85% ethanol solution (1 mL) of potassium carbonate (1 mg) and Kryptofix-2.2.2 (10 mg) without azeotropic drying. The precursor was then added to the reactor in a solution of 15% water 85% ethanol (1 mL) and heated at 100° C. for 30 min. Protected [¹⁸F]fludeoxyglucose was produced in a radiochemical yield (RCY) of 63% (non-decay corrected yield at end-of-synthesis based upon [¹⁸F]fluoride).

### Example 8

### Manual Synthesis of [¹⁸F]Fludeoxyglucose using Non-Dried Fluoride

[¹⁸F]Fludeoxyglucose was prepared by the general procedure for manual synthesis of ¹⁸F-labeled compounds. In this case the [¹⁸F]Fluoride was eluted from a QMA cartridge into the reaction vessel using 15% water 85% ethanol solution of potassium carbonate (1 mg) and Kryptofix-2.2.2 (10 mg) without azeotropic drying. [¹⁸F]Fluoride was combined with a solution of precursor in 15% water:85% ethanol (1 mL) and heated at 100° C. for 30 min. Protected [¹⁸F]fludeoxyglucose was produced in a radiochemical yield (RCY) of up to 42% (n=8) (non-decay corrected yield at end-of-synthesis based upon [¹⁸F]fluoride) as summarized in Table 2.

TABLE 2

| Solvent | Precursor Amount | Reaction Condition | Yield |
|---|---|---|---|
| 15% H₂O/85% EtOH | 40 mg | 100° C., 30 min | 42% |
| 25% H₂O/75% EtOH | 40 mg | 100° C., 30 min | 11% |
| 50% H₂O/50% EtOH | 40 mg | 100° C., 30 min | 14% |
| 75% H₂O/25% EtOH | 40 mg | 100° C., 30 min | 7% |
| 100% H₂O/0% EtOH | 40 mg | 100° C., 30 min | 2% |

### Example 9

### Automated Synthesis of [¹⁸F]Fluoroethyl Tosylate using Dried Fluoride

[¹⁸F]Fluoroethyl tosylate was prepared by the general procedure for automated synthesis of ¹⁸F-labeled compounds described in Example 6 with 1500 mCi initial activity of [¹⁸F]. Under automated conditions, ditosyl methane was converted to [¹⁸F]fluoroethyl tosylate in a radiochemical yield (RCY) of 50% (non-decay corrected yield at end-of-synthesis based upon [¹⁸F]fluoride). The reaction was performed in 100% ethanol at 120° C. for 10 min.

The present method employs water and ethanol as the only solvents for synthesis, module cleaning, synthesis, purification, and reformulation of ¹⁸F-radiopharmaceuticals. The resulting doses are suitable for human administration. The elimination of all other organic solvents from the process simplifies radiopharmaceutical production and QC because the need to purchase, handle, and properly dispose of other hazardous solvents is obviated, and relegates residual solvent analysis from a daily QC requirement to a quarterly or annual requirement. The present methods offers time and economy advantages over other more traditional syntheses, most noticeably in the synthesis of ¹⁸F-FDG.

The present fluorination reactions with fluoride-18 proceed solely in ethanol and water, allowing all other organic solvents to be eliminated from radiosynthesis. This present invention expands the range of solvents that are compatible with fluorine-18 radiochemistry, and simplifies the manufacture of ¹⁸F-radiopharmaceutical doses.

What is claimed:

1. A method of preparing an ¹⁸F-radiopharmaceutical comprising:
(a) providing a mixture of an ¹⁸F-containing salt and a crown ether;
(b) optionally drying the mixture of (a) using a water-ethanol azeotrope; and
(c) reacting the product of (b) with a halide or sulfonate-containing pharmaceutical, wherein a solvent used in the step (c) consists essentially of water, ethanol, or a mixture thereof,
to provide the ¹⁸F-radiopharmaceutical.

2. The method of claim 1 wherein the solvent of step (c) consists essentially of 75% to 95%, by volume, ethanol and 5% to 25%, by volume, water.

3. The method of claim 1 wherein the solvent of step (c) consists essentially of 80% to 90%, by volume, ethanol and 10% to 20%, by volume, water.

4. The method of claim 1 wherein the solvent of step (c) consists essentially of 82% to 90%, by volume, ethanol and 10% to 18%, by volume, water.

5. The method of claim 1 wherein the solvent of step (c) is water.

6. The method of claim 1 wherein the ¹⁸F-radio pharmaceutical is ¹⁸F-labeled fludeoxyglucose, flubatine, fluoropropylcarbomethoxytropane (FP-CT), fluorothymidine, fluorocholine, fluoroethylcholine, fluoropropylcholine, fluoro-2-dialkylamino-6-acylmalonodinitrile naphthalene (FDDNP), fluoroisonidazole, fluoroestradiol, or fluoroethyl tosylate.

7. The method of claim 1 wherein the solvent used in step (c) consists of water and ethanol.
